(19) United States
(12) Patent Application Publication
(10) Pub. No.: US 2006/0089596 A1
(43) Pub. Date: Apr. 27, 2006

(54) Inflatable intraluminal molding device with means for delivering therapy and method of use thereof

(76) Inventor: Biagio Ravo, Rome (IT)

Correspondence Address: DEWITT ROGGIN PLLC, 12 E. LAKE DRIVE, ANNAPOLIS, MD, 21403, US

(21) Appl. No.: 11/299,425

(22) Filed: Dec. 12, 2005

Related U.S. Application Data
(63) Continuation-in-part of application No. 10/825,544, filed on Apr. 15, 2004, now Pat. No. 6,974,441.
Application No. 10/825,544 is a continuation of application No. 09/245,788, filed on Feb. 5, 1999, now abandoned.
(60) Provisional application No. 60/073,902, filed on Feb. 6, 1998.

(51) Int. Cl. A61M 29/00

(52) U.S. Cl. 604/101.01; 600/4

(57) ABSTRACT

An intraluminal molding device in accordance with the disclosed invention has an elongated body member having a series of independent inflatable sections along its length. As lest one inflating/deflating line extends from each inflatable section to a distal end of the body member. The device may be used for positioning, isolating or identifying a lumen by giving shape to a collapsed viscus. The device may also be sued to visualize intraluminal structure or control pressure within a hollow viscus.

### CROSS-REFERENCE TO RELATED APPLICATIONS

The present application is a continuation-in-part of U.S. patent application Ser. No. 10/825,544 filed on Apr. 15, 2004, which was a continuation of U.S. patent application Ser. No. 09/245,788 filed on Feb. 5, 1999, which claimed the benefit of the filing date of U.S. 60/073,902 filed on Feb. 6, 1998. Each of these aforementioned applications is hereby incorporated by reference in its entirety.

### STATEMENT REGARDING FEDERALLY SPONSORED RESEARCH OR DEVELOPMENT

Not applicable.

### FIELD OF THE INVENTION

The present invention relates to the field of intraluminal medical treatment, and more specifically, to an intraluminal molding device for positioning, isolating and/or identifying relevant segments of a hollow viscus during surgical, diagnostic or other procedures.

### BACKGROUND OF THE INVENTION

During operations on any hollow viscus, such as colorectal surgery, gastrointestinal surgery, urological or biliary surgery, it is often necessary to position, isolate and/or identify relevant segments of the hollow viscus being operated upon. It would be advantageous during such surgery, or during any procedure (surgical, diagnostic, theraputical or otherwise) to perform the positioning, isolation and/or identifying procedure from the interior of the hollow viscus.

### SUMMARY OF THE INVENTION

The present invention provides an inflatable intraluminal device for positioning, isolating, identifying, and/or treating a hollow viscus for use internally of the hollow viscus to give shape to the collapsed viscus by acquiring its distended form. The present invention further provides an intraluminal surgical and diagnostic device independently operable at several locations within a hollow viscus. The present invention further provides a device to maintain the lumen of the hollow viscus clear of fluid and/or air. The present invention further provides a device which can assist in visualizing intraluminal structure and control pressure within an internal viscus. The present invention further provides an intraluminal surgical, theraputical and diagnostic device which is easy to manufacture and simple to use and operate.

The present invention comprises an elongated body member with a plurality of independent inflatable balloon sections extending along the length of the body member, and a mechanism for independently inflating each individual balloon section along the length of the body member.

The elongated body member may be in the form of a sleeve which is adapted to fit over an existing scope and/or suction tube. The mechanisms for individually inflating the specific balloon sctions may be formed as individual air/fluid lines extending from each balloon section along the body member to a distal end of the body member. The individual balloon sections may be formed such that they conform to the specific anatomical structure when inflated. One or more suction tubes and/or optical scopes may be positioned at various locations along the body member between adjacent balloon sections. Leads from the individual scopes and suction tubes will extend along the body member to the distal end of the device for appropriate connection to a suction source or video display equipment as known in the art. The suction tubes may also be used to supply medicine or irrigation fluid to selected portions of the hollow viscus.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description, or may be learned by practice of the invention.

### BRIEF DESCRIPTION OF THE DRAWINGS

The invention of the present application will now be described in more detail with reference to preferred embodiments of the architecture and method, given only by way of example, and with reference to the accompanying drawings, in which:

FIG. 1 schematically illustrates an inflatable intraluminal surgical device according to a first embodiment of the present invention.

FIG. 2 schematically illustrates an inflatable intraluminal surgical device according to a second embodiment of the present invention.

FIG. 3 schematically illustrates an inflatable intraluminal surgical device according to a third embodiment of the present invention.

### DESCRIPTION OF THE PREFERRED EMBODIMENTS

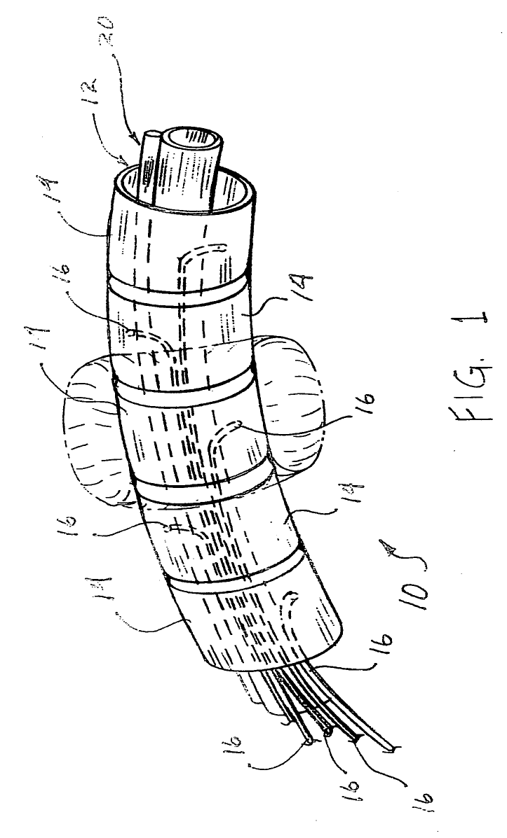

FIG. 1 schematically illustrates an inflatable intraluminal surgical and diagnostic device 10 according to the present invention. The surgical device 10 includes an elongated body member formed as a sleeve 12. along the length of the sleeve 12 are a plurality of independent balloon sections 14. Individual air lines 16 extend from each balloon section 14 along the sleeve 12 to the distal control end of the device 10. the individual air lines 16 may be color coded, numbered or include other appropriate indicia to indicate which specific balloon section 14 along the length of sleeve 12 the specific air line 16 is associated with.

The sleeve 12 is adapted to fit around an existing suction tube and scope member 20. the leading end of the sleeve 12 may include appropriate conventional connecting mechanisms to attach the leading end of the sleeve 12 to the leading end of the suction tube and scope member 20 in the position generally shown in FIG. 1.

In operation, the surgical device 10 is positioned on the suction tube and scope member 20 by sliding sleeve 12 along the suction tube and scope member 20 and attaching the leading end of the sleeve 12 to a leading end of the suction tube and scope member 20 through a connecting mechanism. The surgical device 10, along with the existing suction tube and scope member 20, can be inserted into the specific hollow viscus, such as the colon or esophagus. With the surgical device inserted into the colon, for example, a desired segment of the colon can be isolated by inflating the specific balloon section 14 associated with that segment. The balloon section 14 can be inflated with air, fluid, and/or plasma injected through the appropriate air line 16. The injection may be through the use of a syringe or any appropriate inflation device. The balloon sections 14 may also be deflated through use of air lines 16. the inflated balloon section 14 will expand the colon section serving to isolate and identify the specific section. The segment associated with the inflated balloon section 14 (one of which is shown in phantom in FIG. 1) can be easily identified and positioned from the exterior for appropriate operations. The surgical device 10 may be utilized for isolating a section of a hollow viscus to be operated upon, such as a section of colon to be resected, which is between a pair of inflated, spaced balloon sections 14 along the surgical device 10. The surgical device 10 may also be used to control bleeding along the length of a hollow viscus. There are many independent applications for the surgical device 10 of the present invention.

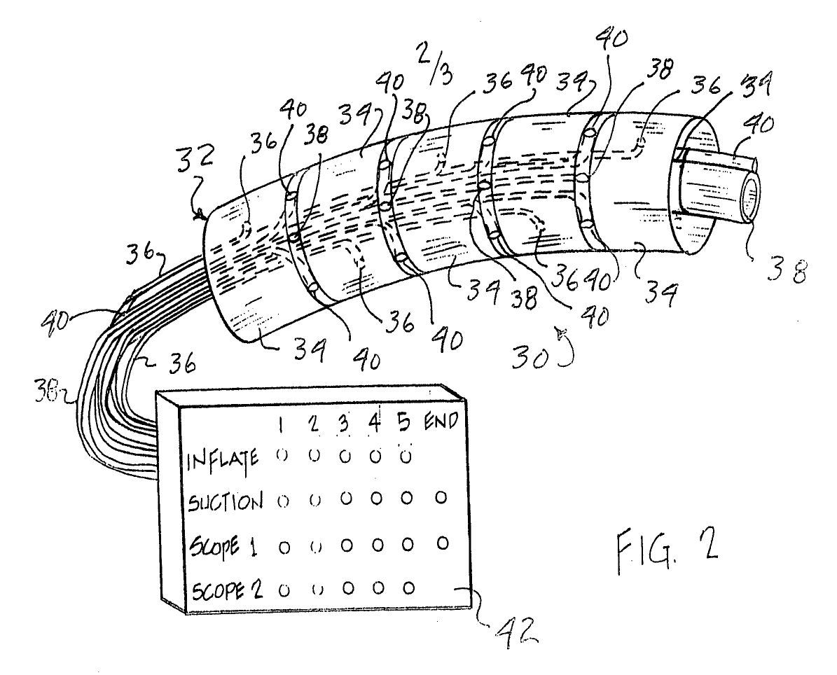

FIG. 2 schematically illustrates a surgical device 30 according to a second embodiment of the present invention. The surgical device 30 is similar to the surgical device 10 and includes a body member 32 with a plurality of independently inflatable/deflatable balloon sections 34 along the length of body member 32. Individual air lines 36 extend from each balloon section 34 along the body member 32. the surgical device 30 additionally includes suction tubes 38 extending from the leading end of the body member 32 and also positioned between adjacent balloon sections 34. the surgical device 30 further includes optical fibers 40 extending from the leading end of the body member 32 and also positioned between adjacent balloon sections 34. The optical fibers 40 between adjacent balloon sections are illustrated extending in opposite directions relative to the body member 32 to provide a comprehensive view to the surgeon or other operator. The air lines 36, suction tubes 38 and optical fibers 40 extend along the body member 32 to an external control panel 42. The control panel 42 allows the specific air lines 36, suction tubes 38 or optical fibers 40 to be accessed as desired.

In operation, the surgical device 30 operates similar to the surgical device 10 above with the surgical device 30 inserted into an appropriate hollow viscus, such as a colon, gastrointestinal track, or the like. The specific balloon sections 34 can be independently inflated and deflated by accessing the associated air line 36 on the control panel 42. As discussed above, the balloon section 34 can be filled with air through air line 36 or fluid, such as saline or the like, through air line 36. Additionally, suction tubes 38 allow for specific sections along the length of the surgical device 30 to be suctioned or for medication or other fluid to be introduced to specific sections along the length of the surgical device 30.

For example, a pair of spaced balloon sections 34 may be inflated to isolate a colon section therebetween which is to be filled with a specific liquid applied through appropriate suction tube 38. This may for example be used to treat a specific isolated section of the colon with chemotherapy. The surgeon can view the desired location along the surgical device 30 by connecting the appropriate optical fibers 40 to visual equipment through connections to control panel 42 in a known manner. As discussed above in connection with surgical device 10, there are many applications, surgical, diagnostical and theraputical for the surgical device 30 of the present invention.

Similarly, the invention may be used to treat specific sections or segments of the hollow viscus. Such a treatment may be performed, for example, by injecting a radiotheraputical compound into a specific balloon section. Other examples that would be apparent to those of skill in the art would be the injection of radiotheraputical, chemotherapeutical or diagnostic materials in between balloon sections, on the outside of balloon section or in balloon section. Consider three adjacent balloon sections. If the two outer balloon sections are inflated and the middle section is not, a theraputical fluid may be injected between the two inflated balloon sections such that it coats the outer surface of the middle balloon section. If the middle balloon section is then inflated, the theraputical fluid will be applied to the section of the colon surrounding that middle balloon section.

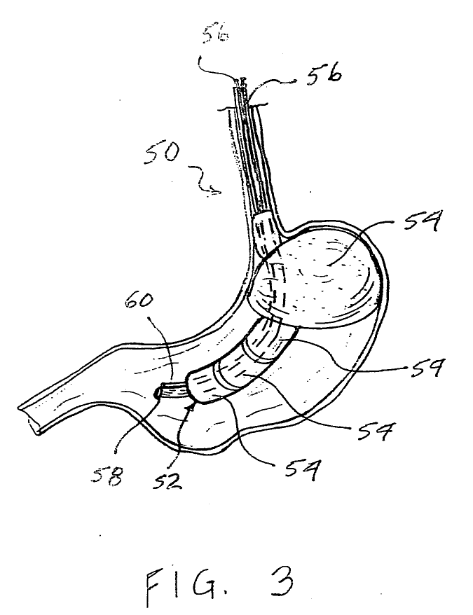

FIG. 3 schematically illustrates a surgical device 50 according to a third embodiment of the present invention. Surgical device 50 is substantially similar to surgical devices 10 and 30 described above and includes an elongated body member 52 with a plurality of spaced balloon sections 54 along the length thereof. Individual air lines 56 extend to these individual balloon sections 54 along the body member 52 in substantially the same manner discussed above in connection with surgical devices 10 and 30. the surgical device 50 is illustrated with a suction tube 58 and an optical fiber 60 extending to the end of the surgical device 50 and may further include suction tubes and optical fibers with the air lines 56 and suction tubes and optical fibers attached to a control panel similar to that discussed above in connection with surgical device 30. the surgical device 50 is designed for a gastrointestinal operation and illustrates that the specific balloon sections 54, when inflated, are designed to conform to the specific anatomical structure. As discussed above, the surgical device 50 of the present invention can be utilized in a wide variety of procedures, both surgical and diagnostic. The device 50 can maintain the desired size and intraluminal pressure on the esophagus eliminating other tubes and dilators currently used.

Any of the devices 10, 30 and 50 of the present invention can be used to help identify lumen perforations, isolate lumen segments, control lumen pressure, control and prevent bleeding into the lumen, medicate and/or suction selected portions of the lumen, visualize inner lumen structure and a variety of other applications. Further, it would be apparent to those of ordinary skill in the art that various modifications may be made to the present invention without departing from the spirit and scope thereof. For example, the devices 10, 30 and 50 may be used with a reusable light source to assist the operation.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. An apparatus comprising:
an elongated body member;
a plurality of inflatable balloons along a length of said body member; and
an inflation/deflation line associated with each of said plurality of balloons; and
means for injecting a radiotheraputical compound into at least one of said plurality of balloons.

2. An apparatus according to claim 1 further comprising:
at least one optical fiber for viewing tissue to be treated.

3. A method of treating a hollow viscus comprising the steps of:
inflating a first balloon within a first section of said hollow viscus to give form to said first section;
inflating a second balloon within a second section of said hollow viscus to give form to said second section;
injecting a theraputical fluid into a third section of said hollow viscus between and adjacent to said first and second balloons;
inflating a third balloon adjacent to both said first balloon and said second balloon to apply said theraputical fluid to substantially all of said third section of said hollow viscus.
